(19) United States
(12) Patent Application Publication
(10) Pub. No.: US 2011/0306920 A1
(43) Pub. Date: Dec. 15, 2011

(54) MEDICAL DEVICE AND METHOD FOR INCREASING ANTI-OXIDATION CAPABILITY

(75) Inventors: Yung-Sheng Lin, Taichung (TW); Ting-Kai Leung, Taichung (TW)

(73) Assignee: HUNGKUANG UNIVERSITY, Taichung (TW)

(21) Appl. No.: 12/946,013

(22) Filed: Nov. 15, 2010

(30) Foreign Application Priority Data
Jun. 10, 2010 (TW) 099118966

(51) Int. Cl. A61N 5/08; C12N 13/00

(52) U.S. Cl. 604/20; 435/173.8

(57) ABSTRACT

A medical device and a method of increasing an anti-oxidation capability of an animal cell are provided. The method of increasing the anti-oxidation capability of the animal cell comprises steps of providing the animal cell, and disposing a far-infrared (FIR) emitter in a certain distance from the animal cell. Particularly, the FIR emitter includes a mineral oxide.

### FIELD OF THE INVENTION

The present invention relates to a medical device and a method for increasing an anti-oxidation capability, and more particularly to a medical device and a method for increasing an anti-oxidation capability of an animal cell.

### BACKGROUND OF THE INVENTION

Oxidative stress results from an imbalance between the production of oxidative free radicals and defense of antioxidant. Recently, many researches have demonstrated that many diseases may result from the oxidative stress. When the oxidative stress is high over, which causes the defense system of body to have no ability to resist, balance or modulate the oxidative stress, the body will be damaged.

Although various antioxidant enzymes may be naturally produced in human body, the sensitivity to the oxidative stress is various between different subjects due to the differences of eating habits, life styles and genetic factors. Therefore, it is necessary to properly strengthen the defense ability against oxidative free radicals in the body.

Common ways of strengthening the defense ability against oxidation includes the supplement of antioxidants. However, it is to be noted that there exists difference of bio-availability of antioxidants between different subjects. Furthermore, either natural or synthesized antioxidants have a defect that the stability thereof is difficult to be maintained. Accordingly, there is need of more stable, convenient and effective ways to strengthen anti-oxidation ability.

### SUMMARY OF THE INVENTION

Far-infrared (FIR) ray at 4˜14 μm is also known as “life wave”. In the present invention, an FIR emitter is used to develop a method to resist the oxidative stress. The FIR emitter can continuously and spontaneously emit an FIR ray, and act without directly contacting with the subject. Therefore, the method provided in the present invention is suitable to be applied in daily life, and is a novel, safe and effective way to strengthen the defense ability against oxidation.

Although materials emitting FIR have been popularly studied and applied, e.g. enhancing the blood circulation via the warming function, there is no study of the effect of FIR upon the animal subject under the oxidative stress yet.

Organisms to be in an oxidation environment would result in the accumulation of substances poisonous to cells, which would directly or indirectly oxidize intracellular bio-molecules, such as nucleic acid and protein, and cause the cells to be damaged and further to accelerate the aging and apoptosis of the cells. Moreover, in view of the defects of prior antioxidants, which are difficult to be preserved, and apt to be destroyed and then lose efficacy when being exposed to certain environments such as light and heat source, a physical method of anti-oxidation is provided to continuously and non-contactly enhance anti-oxidation ability of the subject. The methods provided in the present invention are compatible with various situations, avoid the application limit of the prior arts, and thus have significant development values.

In accordance with one aspect of the present invention, a method of increasing an anti-oxidation capability of an animal cell is provided. The method comprises steps of providing the animal cell, and disposing a far-infrared (FIR) emitter in a certain distance from the animal cell.

Preferably, the FIR emitter includes a mineral oxide.

Preferably, the animal cell exists in an oxidation environment having a peroxide.

Preferably, the anti-oxidation capability of the animal cell includes an ability being one selected from a group consisting of promoting a survival of the animal cell, reducing the peroxide, avoiding an apoptosis, preventing an oxidative-reduction imbalance of the peroxide, and a combination thereof.

Preferably, the animal cell includes one selected from a group consisting of a fibroblast, an osteoblast and a macrophage.

Preferably, the FIR emitter has a radiation range to emit an FIR ray, and the certain distance is one of distances lower than and equal to the radiation range.

Preferably, the FIR emitter has an emissivity higher than 0.9 when the FIR ray emitted thereby has a wavelength ranged from 6 to 14 micrometers.

Preferably, the mineral oxide includes an aluminum oxide (Al₂O₃) ranged from 60% to 95% by weight.

Preferably, the mineral oxide further includes one selected from a group consisting of an iron oxide (Fe₃O₄), a magnesium oxide (MgO), a zinc oxide (ZnO), a calcium carbonate (CaCO₃) and a combination thereof.

Preferably, the mineral oxide includes Al₂O₃, Fe₃O₄, MgO, ZnO and CaCO₃, which have a weight ratio of 90:2:5:2:1.

In accordance with another aspect of the present invention, a method of providing a medical effect being one selected from a group consisting of promoting a healing of a wound, promoting a bone formation and enhancing an immunity is provided. The method comprising steps of providing a subject in need thereof, and disposing a mineral oxide in a certain distance from the subject.

Preferably, the subject is one of an animal.

Preferably, the subject is an animal cell being one selected from a group consisting of a fibroblast, an osteoblast and a macrophage.

Preferably, the animal cell exists in an oxidation environment having a peroxide.

Preferably, the mineral oxide includes an alumina oxide ranged from 60% to 95% by weight.

In accordance with a further aspect of the present invention, a medical device for increasing an anti-oxidation capability of an animal cell is provided. The medical device comprises a far-infrared (FIR) emitter including a mineral oxide and disposed in a certain distance from the animal cell.

Preferably, the FIR emitter has a radiation range to emit an FIR ray, and the certain distance is one of distances lower than and equal to the radiation range.

Preferably, the FIR emitter has an emissivity higher than 0.9 when the FIR ray emitted thereby has a wavelength ranged from 6 to 14 micrometers.

Preferably, the FIR emitter is configured in a form being one selected from a group consisting of a patch, a clothes, a wrist support, a waist support, a knee support, a mattress and a pillow.

Preferably, the mineral oxide includes an aluminum oxide ranged from 60% to 95% by weight.

Preferably, the mineral oxide includes Al₂O₃, Fe₃O₄, MgO, ZnO and CaCO₃, which have a weight ratio of 90:2:5:2:1.

The above objects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 is a diagram showing the effect of FIR on osteoblastic cell viability in H₂O₂-induced cytotoxicity.

FIG. 2 is a diagram showing the effect of FIR on fibroblastic cell viability in H₂O₂-induced cytotoxicity.

FIG. 3(A) is a diagram showing extracellular H₂O₂ level of RAW264.7 cells without LPS induction, after being effected by FIR in 1 day.

FIG. 3(B) is a diagram showing extracellular H₂O₂ level of RAW264.7 cells without LPS induction, after being effected by FIR in 2 days.

FIG. 4 is a diagram showing extracellular H₂O₂ level of RAW264.7 cells with LPS induction, after being effected by FIR in 1 day.

FIG. 5 is a diagram showing cell viability of RAW264.7 cells treated with FIR under oxidative stress.

FIG. 6(A) is a diagram showing result of the effect of FIR on apoptosis of RAW264.7 cells induced by H₂O₂.

FIG. 6(B) is a diagram showing result of the effect of FIR on LDH release of RAW264.7 cells.

FIG. 7 is a diagram showing the effect of FIR on intracellular H₂O₂ level of RAW264.7 cells.

FIG. 8 is a diagram showing the effect of FIR on intracellular cytochrome c level of RAW264.7 cells.

FIG. 9 is a diagram showing the effect of FIR on NADP⁺/NADPH of RAW264.7 cells.

### DETAILED DESCRIPTION OF THE PREFERRED EMBODIMENT

The present invention will now be described more specifically with reference to the following embodiments. It is to be noted that the following descriptions of embodiments of this invention are presented herein for the purposes of illustration and description only; it is not intended to be exhaustive or to be limited to the precise form disclosed.

### Example

An embodiment of the FIR emitter according to the present invention is FIR ceramic powder (e.g. 20 g) including Al₂O₃, Fe₃O₄, MgO, ZnO and CaCO₃, which have a weight ratio of 90:2:5:2:1. For example, the amount of Al₂O₃ is about 90% of the total weight of the FIR emitter (i.e. wt %). The mentioned FIR ceramic powder is enclosed in plastic bags as the FIR irradiation source, and the following activity testing assays are performed to analyze the use of antioxidation of the FIR emitter in the present invention.

Activity Testing

Reactive oxygen radicals of H₂O₂ are important factors of oxidative stress related to the pathogenesis of many important diseases in organisms. Cumulative increases in H₂O₂ and superoxide radicals can potentially damage cells including proteins, lipids, and DNA leading to proven augmented mutation rates. Accordingly, H₂O₂ is utilized to simulate an environment having oxidative stress in the present invention.

In pathophysiological conditions, H₂O₂ is continuously generated, and its level remains higher than normal. Exposure of cells to H₂O₂ for a certain period may lead to cell death by apoptosis and necrosis. Cell death or cytotoxicity is classically evaluated by quantifying cytoplasm or plasma membrane damage. In the other hand, because H₂O₂ is highly membrane-permeable, the equilibrium between extra- and intracellular levels of H₂O₂ can be reached extremely rapidly when H₂O₂ gradients form since the H₂O₂ production site and H₂O₂ degradation site are separated by membranes, such that the measurement of intracellular levels of H₂O₂ also reflects extracellular levels thereof.

Cell Viabilities of Osteoblast and Fibroblast

It has been reported that the oxidative stress induced by H₂O₂ is one of the main factors in causing osteoblastic cells death. The reducing of the osteoblastic cells will result in significant decreasing of bone formation and cause bone loss. Under the influence of aging, osteoblastic cells will be more easily to be damaged by the oxidative stress. This also explains the reason why postmenopausal women and aged person are easy to suffer a fracture. Therefore, the analysis of the viability of osteoblastic cells is used as an indication that the FIR emitter can promote a bone formation.

It has been reported that peroxidative ions produced by ultraviolet (UV) rays would be converted to H₂O₂, which inhibits the viability of epidermal fibroblastic cells. Fibroblastic cells are necessary for wound healing due to the capability of resisting the oxidation damage induced by H₂O₂. In the treatment for a burned wound or an epidermal injury, anti-oxidation agent or radical scavenger is helpful to healing process. Therefore, the analysis of the viability of fibroblastic cells is used as an indication that the FIR emitter can promote a healing of a wound.

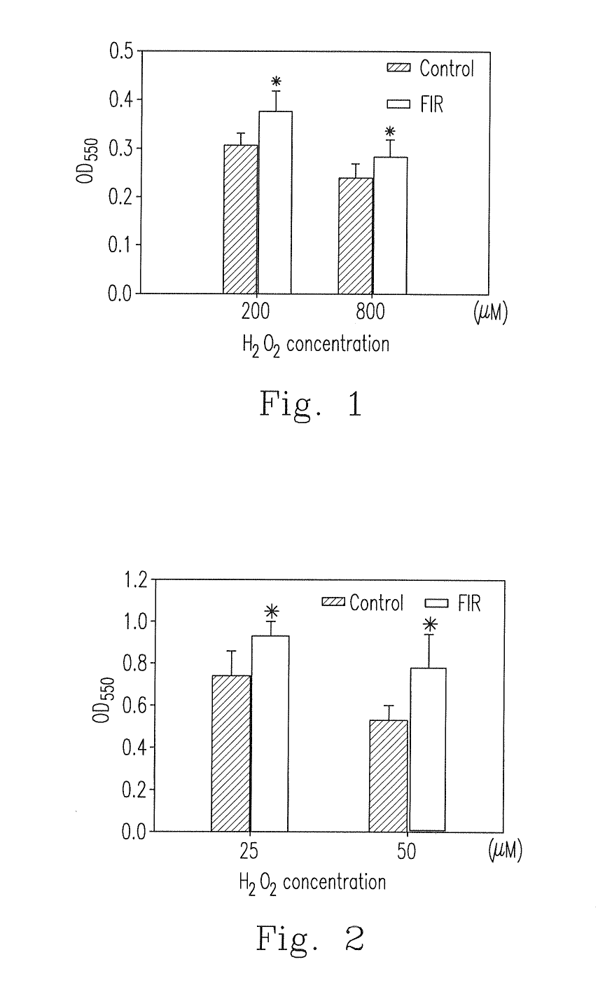

The analyses of cell viabilities of osteoblast and fibroblast under oxidative stress induced by H₂O₂ are performed by MTT assay, respectively. Please refer to FIGS. 1 and 2, which respectively shows the effect of FIR on viabilities of osteoblastic cells and fibroblastic cells in H₂O₂-induced cytotoxicity. As the results shown in FIGS. 1 and 2, the viabilities of cells treated with FIR under H₂O₂-mediated oxidative stress is higher than the control counterparts which receive H₂O₂ treatment without FIR. In fact, additional FIR treatment for the osteoblastic cells in H₂O₂-induced cytotoxicity results in about 23.02% cell viability increases in the 200 μM H₂O₂ concentration group, and results in about 18.77% cell viability increases in the 800 μM H₂O₂ concentration group. Furthermore, additional FIR treatment for the fibroblastic cells in H₂O₂-induced cytotoxicity results in about 25.67% cell viability increases in the 25 μM H₂O₂ concentration group, and results in about 47.16% cell viability increases in the 50 μM H₂O₂ concentration group. The t-test (n=28) further confirms that the cells with FIR treatment may suffer higher H₂O₂-induced oxidative stress.

Extracellular H₂O₂ Production of RAW264.7 Cells

Macrophages play a significant role in innate immunity and inflammation. When macrophages are activated by pathogens and/or cytokines, they produce large amounts of H₂O₂ and ROS to exert strong cytotoxicity against microorganisms and many cells, including killing macrophages themselves. Therefore, increasing the survival rate of macrophages would enhance cell-mediated immunity. Because macrophages are vital for the recognition and elimination of microbial pathogens, and the survival of macrophages may directly contribute to a host's defense system. Accordingly, RAW264.7 murine macrophages are used as a cell target to analysis the effect of the FIR emitter of the present invention on the bio-activity under oxidative stress.

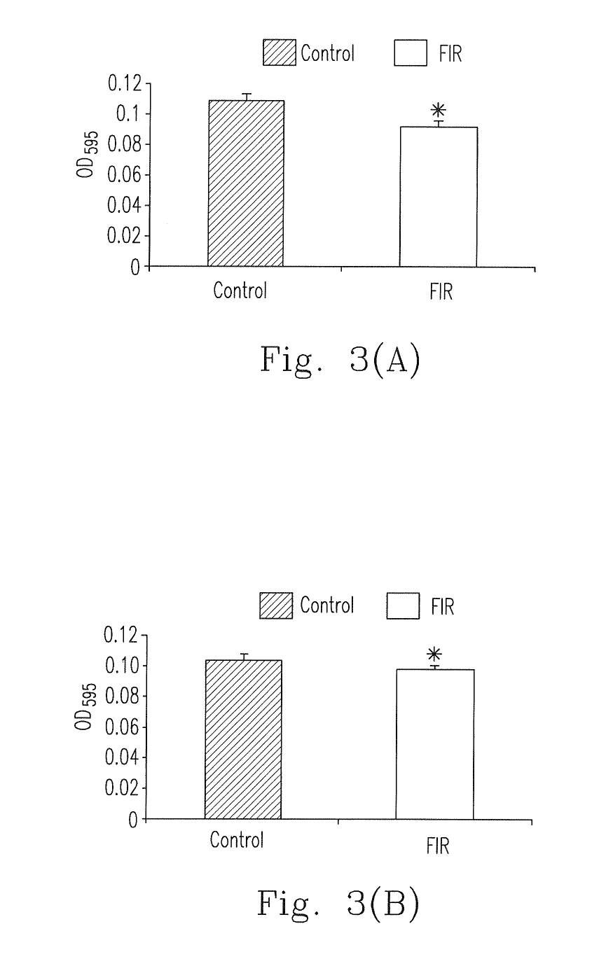

Please refer to FIGS. 3(A) and 3(B), which are diagrams showing extracellular H₂O₂ level of RAW264.7 cells without Lipopolysaccharide (LPS) induction, after being effected by FIR in 1 day and 2 days, respectively, wherein the peroxide level is significantly reduced (*p<0.05).

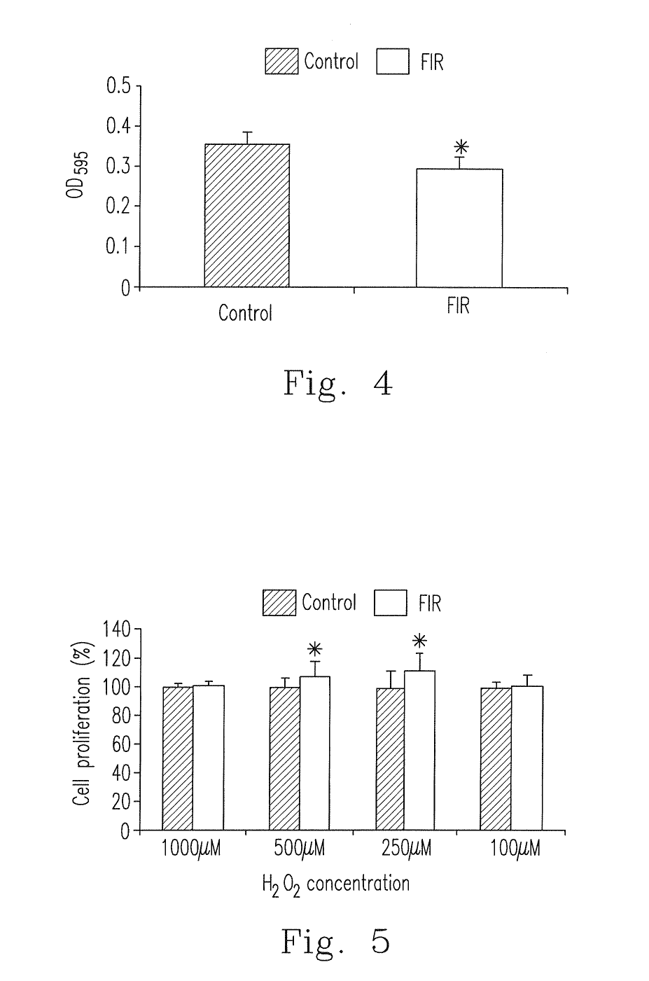

Please refer to FIG. 4, which is a diagram showing extracellular H₂O₂ level of RAW264.7 cells with LPS induction (a simulation of an inflammatory condition in an animal subject). After being effected by FIR in 1 day, the peroxide level is significantly decreased (*p<0.05).

Viability of RAW264.7 Cells

Please refer to FIG. 5, which is a diagram showing cell viability of RAW264.7 cells treated with FIR under oxidative stress, wherein different concentration of H₂O₂ is used as the source of oxidative toxicity, and the cell proliferation (%) of RAW264.7 cells against H₂O₂ is determined via XTT assay. As shown in FIG. 5, FIR possesses ability to decrease the death of RAW264.7 cells under H₂O₂ (*p<0.05).

Hypodiploid Cell Analysis and Lactate Dehydrogenase (LDH) Activity Release Assay

RAW264.7 cells are seeded in 6-well tissue culture plates at a density of 4×10⁵ cells per well. After 24 hours of culturing, the medium is changed and various concentrations of H₂O₂ (400 μM and 600 μM) are added. For FIR groups, enclosed FIR ceramic powder is distributed uniformly in plastic bags, which has been inserted beneath the tissue culture plates. Cells are treated with FIR for a further 24 hours. Cells are washed with PBS and stained with 3 μM propidium iodide (PI; molecular probes) for 30 min. The fluorescence emitted from the PI-DNA complex is quantitated after excitation of the fluorescent dye by FACScan flow cytometry (Becton Dickinson Co.).

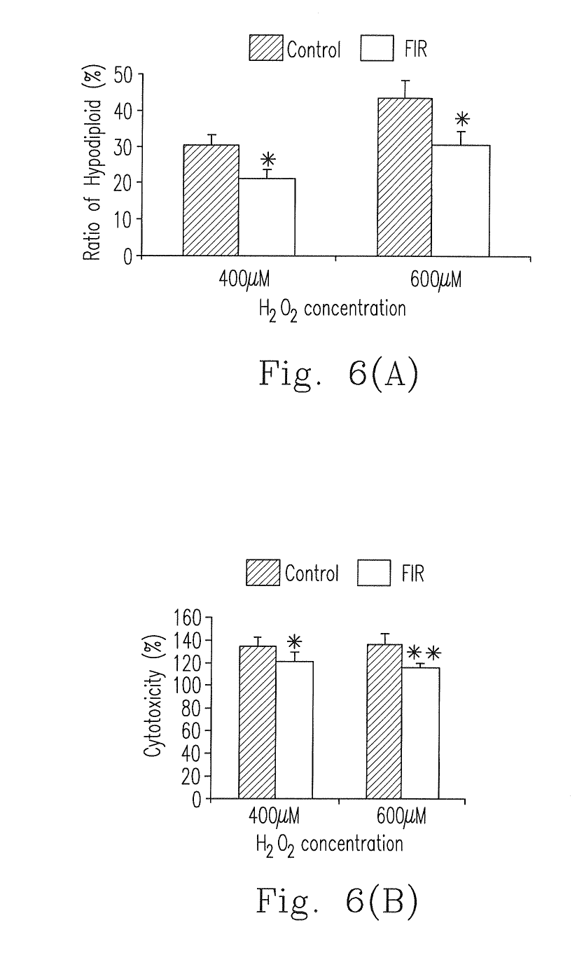

Please refer to FIG. 6(A), which is a diagram showing result of the effect of FIR on apoptosis of RAW264.7 cells induced by H₂O₂. As shown in FIG. 6(A), the ratio of hypodiploid cells increases in H₂O₂-treated cells, and that is significantly reduced after being treated with FIR (*p<0.05), which indicates that FIR can reduce the apoptosis of cells induced by H₂O₂. Please refer to FIG. 6(B), which is a diagram showing result of the effect of FIR on LDH release of cells. As shown in FIG. 6(B), under the induction of H₂O₂ (400 μM and 600 μM), LDH release increases in H₂O₂-treated cells, and that is significantly reduced in FIR group. The effect of FIR on LDH release assays indicates a significant difference between the control and FIR groups for H₂O₂-treated cells (*p<0.05; **p<0.01).

According to FIG. 6(B) of the present application, LDH is utilized to prove the antioxidant ability of cells treated with FIR in the circumstance of H₂O₂ toxicity. LDH is a stable enzyme presenting in all cell types, which is rapidly released into the cell culture medium upon damage to plasma membranes. Therefore, LDH is the most widely used marker in cytotoxicity studies. FIG. 6(B) shows significant reduce of LDH release in cells treated with FIR, which means that the damage to the cells is decreased.

Intracellular H₂O₂ Level of RAW264.7 Cells

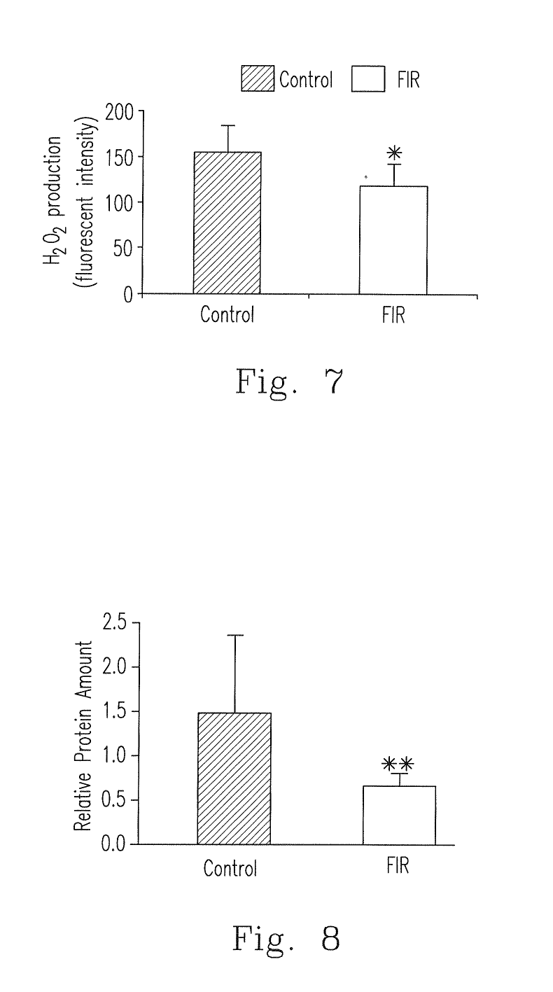

Please refer to FIG. 7, which is a diagram showing the effect of FIR on intracellular H₂O₂ level of RAW264.7 cells. FIG. 7 shows that level of intracellular H₂O₂ is significantly reduced by FIR (compared with the control group, *p<0.05) via flow cytometry analysis using DCHF-DA as a peroxide-sensitive fluorescent dye.

Measurement of the Level of Cytochrome C

Excessive ROS causes degenerative diseases of aging, particularly cancer and atherosclerosis as consequences of oxidative damage by ROS. Protection of cells from such intracellular oxygen radicals appears to be due to the presence of a variety of intracellular enzymes and naturally occurring radical scavengers, such as cytochrome c. Under normal conditions, these protective mechanisms are adequate to prevent extensive damage to vital cellular constituents.

In many aerobic mammalian cells, the generation of oxygen radicals and H₂O₂ in the respiratory chain is a result of electron leakage. Mitochondria represent a primary source of ROS in the cells. Cytochrome c is an ideal antioxidant, which attacks superoxide and oxygen radical. Accordingly, cytochrome c is essential in the respiratory chain for keeping a lower physiological H₂O₂ concentration in mitochondria. Lack of cytochrome c within the respiratory chain causes the higher level of oxygen radicals and associate H₂O₂ accumulation. The level of oxygen radicals and H₂O₂ is in a balanced state between the generation of respiratory chain and the elimination of cytochrome c. Please refer to FIG. 8, which is a diagram showing the effect of FIR on intracellular cytochrome c level of RAW264.7 cells. As shown in FIG. 8, by immunoblotting analysis, the level of cytochrome c in FIR irradiated groups of cells is significantly decreased (**p<0.01), which demonstrates that FIR enhances the antioxidant effect for H₂O₂ by consuming more of intracellular cytochrome c.

Measurement of NADP⁺/NADPH

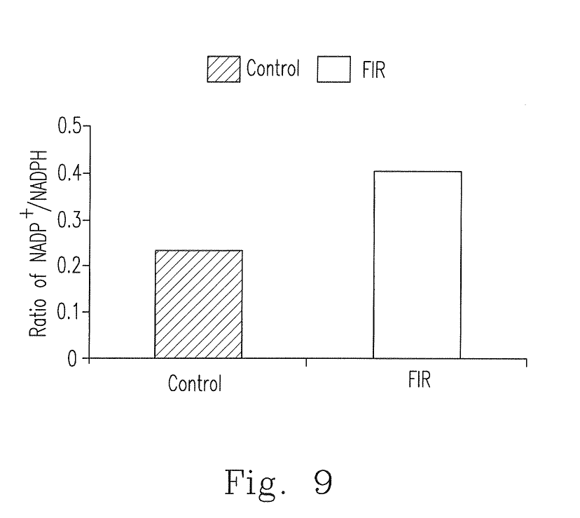

Please refer to FIG. 9, which is a diagram showing the effect of FIR on NADP⁺/NADPH of RAW264.7 cells. The ratio of NADP⁺/NADPH is measured according to the absorbance of the reduced coenzyme at 340 nm assayed by spectrophotometer. As shown in FIG. 9, the ratio is increased in FIR group. By the effect of FIR, more NADPH is consumed and associate increase of NADP⁺, and thus the ratio of NADP⁺/NADPH is increased in FIR group.

This is the first invention disclosing that FIR may exhibit antioxidant characteristics in a mammalian cell via the effects on intracellular level of H₂O₂, cytochrome c and the NADP⁺/NADPH levels. Based on the aforementioned embodiments, a possible pathway that FIR has the antioxidant effect may include the oxidation and electron transfer of NADPH triggered by FIR, decomposition of H₂O₂ into water, and reducing the level of cytochrome c.

According to the aforementioned embodiments, the methods and the medical device provided in the present invention are easy to be applied and configurable in various forms, and can continuously generate desired effect without need of specific or complicate operations. The methods and the medical device provided in the present invention can achieve various effects relating to antioxidation, such as promoting a healing of a wound, promoting a bone formation and enhancing an immunity. Accordingly, the medical device can be configured in any form of a health care product, which includes, but is not limited to, a patch, a clothes, a wrist support, a waist support, a knee support, a mattress and a pillow.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A method of increasing an anti-oxidation capability of an animal cell, comprising steps of:
providing the animal cell; and
disposing a far-infrared (FIR) emitter in a certain distance from the animal cell, wherein the FIR emitter includes a mineral oxide.

2. The method as claimed in claim 1, wherein the animal cell exists in an oxidation environment having a peroxide.

3. The method as claimed in claim 2, wherein the anti-oxidation capability of the animal cell includes an ability being one selected from a group consisting of promoting a survival of the animal cell, reducing the peroxide, avoiding an apoptosis, preventing an oxidative-reduction imbalance of the peroxide, and a combination thereof.

4. The method as claimed in claim 1, wherein the animal cell includes one selected from a group consisting of a fibroblast, an osteoblast and a macrophage.

5. The method as claimed in claim 1, wherein the FIR emitter has a radiation range to emit an FIR ray, and the certain distance is one of distances lower than and equal to the radiation range.

6. The method as claimed in claim 5, wherein the FIR emitter has an emissivity higher than 0.9 when the FIR ray emitted thereby has a wavelength ranged from 6 to 14 micrometers.

7. The method as claimed in claim 1, wherein the mineral oxide includes an aluminum oxide ranged from 60% to 95% by weight.

8. The method as claimed in claim 7, wherein the mineral oxide further includes one selected from a group consisting of an iron oxide, a magnesium oxide, a zinc oxide, a calcium carbonate and a combination thereof.

9. The method as claimed in claim 1, wherein the mineral oxide includes an aluminum oxide, an iron oxide, a magnesium oxide, a zinc oxide and a calcium carbonate, which have a weight ratio of 90:2:5:2:1.

10. A method of providing a medical effect being one selected from a group consisting of promoting a healing of a wound, promoting a bone formation and enhancing an immunity, the method comprising steps of:
providing a subject in need thereof; and
disposing a mineral oxide in a certain distance from the subject.

11. The method as claimed in claim 10, wherein the subject is an animal.

12. The method as claimed in claim 10, wherein the subject is an animal cell being one selected from a group consisting of a fibroblast, an osteoblast and a macrophage.

13. The method as claimed in claim 12, wherein the animal cell exists in an oxidation environment having a peroxide.

14. The method as claimed in claim 10, wherein the mineral oxide includes an alumina oxide ranged from 60% to 95% by weight.

15. A medical device for increasing an anti-oxidation capability of an animal cell, comprising:
a far-infrared (FIR) emitter including a mineral oxide and disposed in a certain distance from the animal cell.

16. The medical device as claimed in claim 15, wherein the FIR emitter has a radiation range to emit an FIR ray, and the certain distance is one of distances lower than and equal to the radiation range.

17. The medical device as claimed in claim 16, wherein the FIR emitter has an emissivity higher than 0.9 when the FIR ray emitted thereby has a wavelength ranged from 6 to 14 micrometers.

18. The medical device as claimed in claim 15, wherein the FIR emitter is configured in a form being one selected from a group consisting of a patch, a clothes, a wrist support, a waist support, a knee support, a mattress and a pillow.

19. The medical device as claimed in claim 15, wherein the mineral oxide includes an aluminum oxide ranged from 60% to 95% by weight.

20. The medical device as claimed in claim 15, wherein the mineral oxide includes an aluminum oxide, an iron oxide, a magnesium oxide, a zinc oxide and a calcium carbonate, which have a weight ratio of 90:2:5:2:1.
